(19) United States
(12) Patent Application Publication
(10) Pub. No.: US 2026/0224250 A1
(43) Pub. Date: Aug. 6, 2026

(54) ORTHOPEDIC ANCHOR SYSTEM

(71) Applicant: GENESYS SPINE, Austin, TX (US)

(72) Inventors: Josh KAUFMANN, Austin, TX (US); Derek SOUTHARD, Austin, TX (US); Landon GILKEY, Farmington, NM (US); Greg CALBERT, Austin, TX (US); Scott BRYANT, Austin, TX (US); Maahir HAQUE, Orlando, FL (US); Simon SALERNO, Holmdel, NJ (US)

(21) Appl. No.: 19/146,293

(22) PCT Filed: Jan. 9, 2024
(86) PCT No.: PCT/US2024/010826

§ 371 Date: Jul. 8, 2025

Related U.S. Application Data
(60) Provisional application No. 63/479,163, filed on Jan. 9, 2023.

(51) Int. Cl. A61B 17/70; A61B 17/86

(52) U.S. Cl. CPC A61B 17/7055; A61B 17/7005; A61B 17/7032; A61B 17/863

(57) ABSTRACT

An embodiment includes a sacroiliac joint bone anchor system comprising: a sleeve that includes: a central sleeve channel, a threaded first portion and a threaded second portion, a third portion, a central sleeve axis, and a plurality of threads; a rod that includes: a central rod channel, a head on the proximal end of the rod, the head including a circular cross-section, a central rod axis, and a plurality of threads on an outer surface of the rod, wherein, the rod is proportioned to slide within the central sleeve channel, and the plurality of threads on the outer surface of the rod are configured to screw into the plurality of threads on the wall of the central sleeve channel.

### CROSS-REFERENCE TO RELATED APPLICATION

This application claims priority to U.S. Provisional Patent Application No. 63/479,163 filed on Jan. 9, 2023 and entitled “Orthopedic Anchor System”, the content of which is hereby incorporated by reference.

### TECHNICAL FIELD

Embodiments of the invention are in the field of orthopedic implants.

### BACKGROUND

The sacroiliac joint (SI joint) is a joint in the pelvis that connects the sacrum and the ilium. When ligaments of the joint become loose or otherwise dysfunctional the SI joint may fail to provide adequate shock absorption for the spine, which may be painful. SI joint fusion is a technique used to stabilize the SI joint and to alleviate pain caused by the dysfunctional joint. One surgical technique includes placing bone graft within the joint and then driving one or more compression screws across the joint to stabilize the joint. This may eventually lead to fusion of the joint and, ideally, less pain.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIGS. 1A, 1B provide differing views of an embodiment.

FIGS. 2A, 2B provide differing views of an embodiment.

FIGS. 3A, 3B, 3C, 3D, 3E provide differing views of an embodiment. FIGS. 3B and 3C cross-sectional views the embodiment of FIG. 3A. FIGS. 3F, 3G, 3H provide differing views of the embodiment of FIG. 3B but with a tulip attached to the head of the rod.

FIGS. 4A, 4B, 4C, 4D provide differing views of an embodiment of the implant included within a fusion system that extends from the SI joint to vertebral members.

FIGS. 5A, 5B provide differing views of an embodiment.

FIGS. 6A, 6B provide differing views of an embodiment.

FIG. 7A shows a perspective view of long and short versions of an embodiment. FIG. 7B shows a cross-sectional view (partial) of the embodiments of FIG. 7A. FIGS. 7C and 7D show perspective views of an embodiment (such as the shorter embodiment of FIG. 7B) including a tulip. FIG. 7E shows a series of short and long embodiments which may be included in a kit.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments. Moreover, the drawings may only show the structures useful to understand the illustrated embodiments. Additional structures known in the art may not have been included to maintain the clarity of the drawings. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical contact with each other and “coupled” may indicate elements co-operate or interact with each other, but they may or may not be in direct physical contact. Phrases such as “comprising at least one of A and B” include situations with A, B, or A and B.

An embodiment provides an anchor. One example of an anchor is a screw. Other examples include nails and the like.

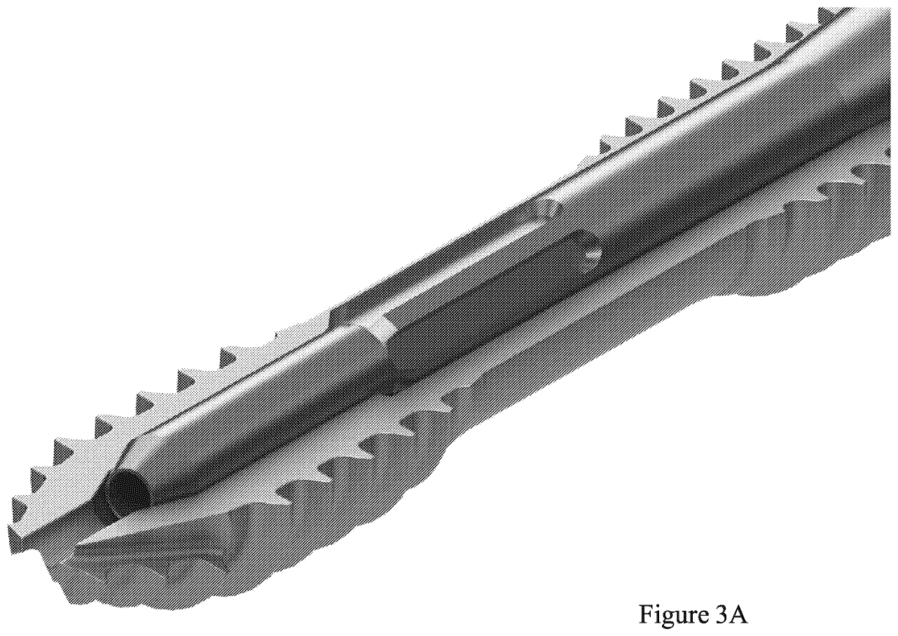

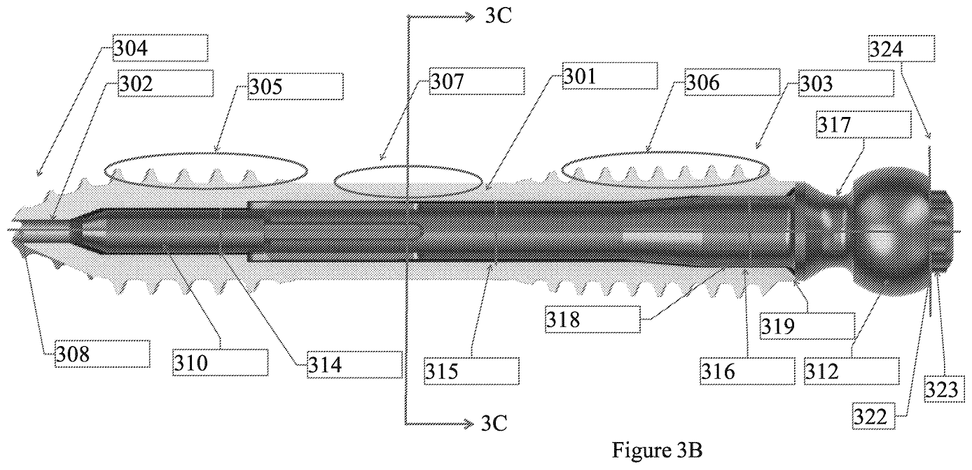

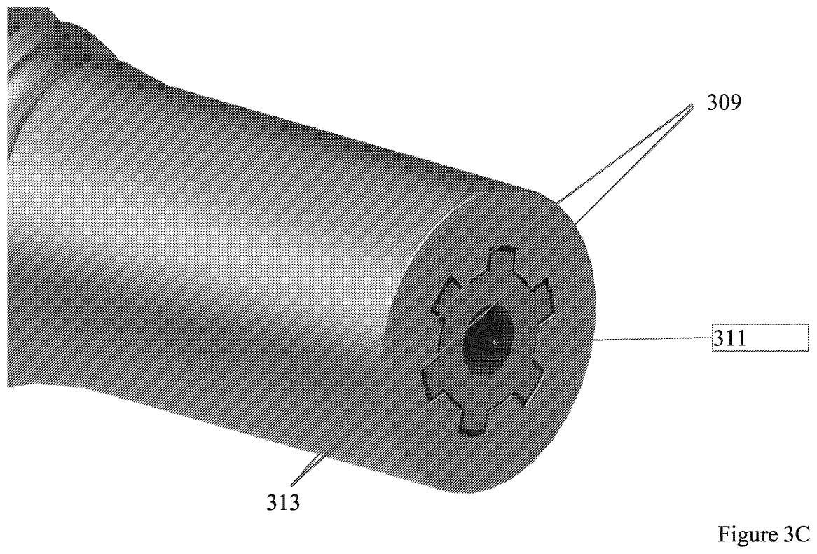

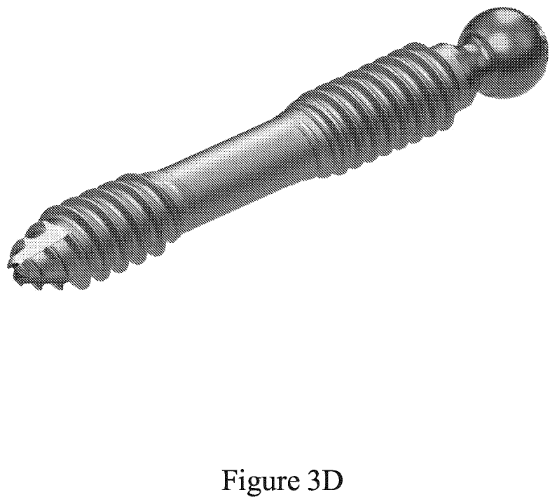

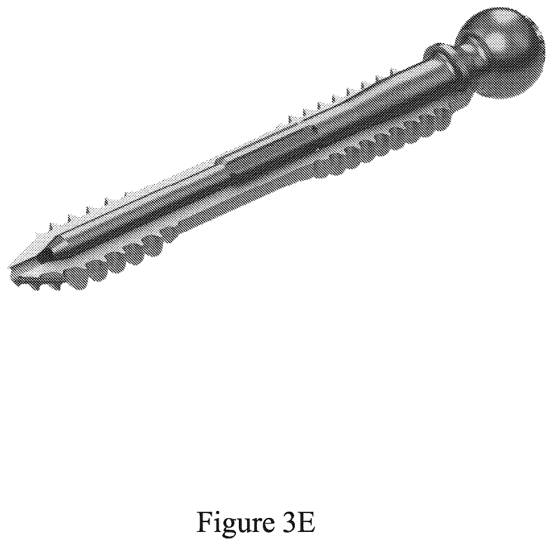

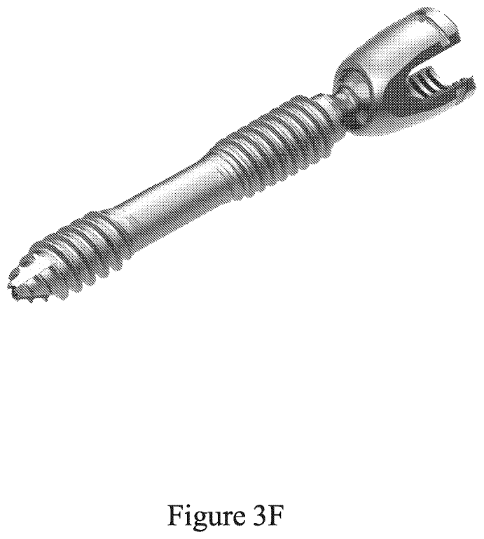

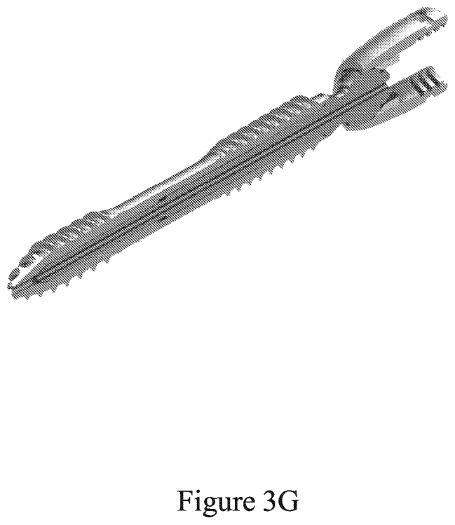

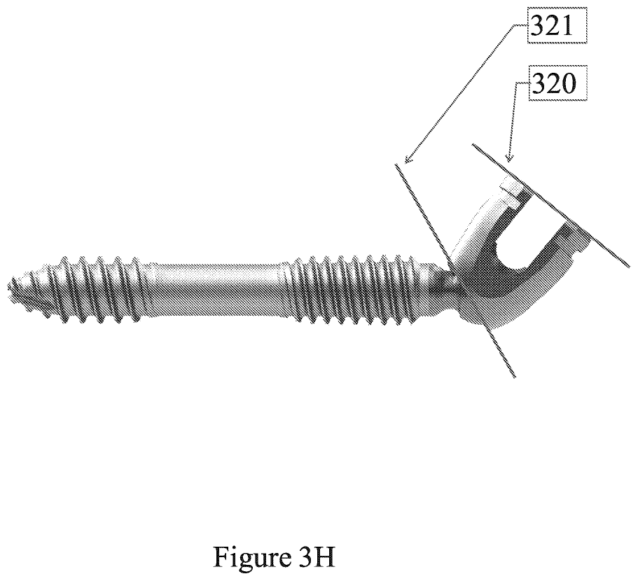

As shown in FIG. 3A-3H, an embodiment includes a bone anchor system comprising a sleeve (301) that includes a central sleeve channel (302) that traverses the sleeve and extends from a proximal end (303) of the sleeve to a distal end (304) of the sleeve. The sleeve further includes first (305) and second (306) threaded portions on an outer surface of the sleeve, and a non-threaded portion (307) on the outer surface of the sleeve and existing between the first and second threaded portions. The sleeve includes a central sleeve axis (308) that extends from the proximal end of the sleeve to the distal end of the sleeve. The sleeve includes a plurality of linear projections (309) on a wall of the central sleeve channel.

The system further includes a rod or shaft (310) that includes a central rod channel (311) that traverses the rod and extends from a proximal end of the rod to a distal end of the rod. The rod includes a head (312) on the proximal end of the rod, the head including a circular cross-section. The rod includes a central rod axis that extends from the proximal end of the rod to the distal end of the rod. The rod includes a plurality of linear slots (313) on an outer surface of the rod. The rod is proportioned to slide within the central sleeve channel, and the plurality of linear projections is proportioned to slide within the plurality of linear slots.

An alternative embodiment includes a bone anchor system comprising a sleeve (301) that includes: (a) a central sleeve channel (302) that traverses the sleeve and extends from a proximal end (303) of the sleeve to a distal end (304) of the sleeve, (b) first (305) and second (306) threaded portions on an outer surface of the sleeve, (c) a central sleeve axis (308) that extends from the proximal end of the sleeve to the distal end of the sleeve, and (e) a plurality of linear projections (309) on a wall of the central sleeve channel. The system further includes a rod (310) that includes: (a) a central rod channel (311) that traverses the rod and extends from a proximal end of the rod to a distal end of the rod, (b) a head (312) on the proximal end of the rod, the head including a circular cross-section; (c) a central rod axis that extends from the proximal end of the rod to the distal end of the rod, and (e) a plurality of linear slots (313) on an outer surface of the rod. The rod is proportioned to slide within the central sleeve channel, and the plurality of linear projections is proportioned to slide within the plurality of linear slots.

Thus, for example, not all embodiments necessarily include non-threaded portion 307. Some embodiments may extend threads 305 or 306 into areas 307. Other embodiments may include a set of threads with a pitch that differs from the pitch of threads of either or both of threads 305, 306.

Fenestrations (i.e., apertures, holes) may be included in smooth or threaded areas of the sleeve and/or rod. In some embodiments, fenestrations are included in both the sleeve and rod such that an axis, which is orthogonal to a long axis of the sleeve, traverse the apertures. Multiple apertures may be included in the rod and sleeve such that the apertures cooperate to provide a continuous void that extends from one side of the sleeve, across the rod, and through the other side of sleeve to provide a path for bone fusion/growth.

An alternative embodiment of the bone anchor system comprises: a sleeve (301) that includes: (a) a central sleeve channel (302) that traverses the sleeve and extends from a proximal end (303) of the sleeve to a distal end (304) of the sleeve, (b) first (305) and second (306) threaded portions on an outer surface of the sleeve, (c) a non-threaded portion (307) on the outer surface of the sleeve and existing between the first and second threaded portions, (d) a central sleeve axis (308) that extends from the proximal end of the sleeve to the distal end of the sleeve, and (e) a plurality of linear projections (309) on a wall of the central sleeve channel. The system includes a rod (310) that includes: (a) a central rod channel (311) that traverses the rod and extends from a proximal end of the rod to a distal end of the rod, (b) a head (312) on the proximal end of the rod, the head including a circular cross-section; (c) a central rod axis that extends from the proximal end of the rod to the distal end of the rod, and (e) a plurality of linear slots (313) on an outer surface of the rod.

In an embodiment, a first of the plurality of the linear projections includes a first projection long axis that is parallel to the central sleeve axis and a second of the plurality of linear projections includes a second projection long axis that is parallel to the central sleeve axis.

In an embodiment, a first of the plurality of the linear slots includes a first slot long axis that is parallel to the central rod axis; and a second of the plurality of linear slots includes a second slot long axis that is parallel to the central rod axis.

In an embodiment, the central sleeve channel includes first (314), second (315), and third (316) inner diameters. The first inner diameter is distal to the second and third inner diameters and the second inner diameter is between the first and third inner diameters. The second inner diameter is less than the third inner diameter. For example, diameter 314 may be less than diameter 315 as a result of forming slots in the rod. In other words, dimeter 314 is the same as the diameter of the rod taken from a slot to another slot on the opposing side of the rod.

In an embodiment the first inner diameter is less than the second inner diameter.

In an embodiment, the rod includes first, second, and third outer diameters. The first outer diameter is distal to the second and third outer diameters and the second outer diameter is between the first and third outer diameters. The second outer diameter is less than the third outer diameter.

In an embodiment, the first outer diameter is less than the second outer diameter. However, in other embodiments the diameter relationships are not so limited and may include more or fewer distinct outer diameters for the rod. For example, diameter 1 (314) may remain but diameter 2 (315) may extend proximally to end 303. In an embodiment, inner diameters for the sleeve correspond to outer diameters for the rod. In other words, the inner and outer diameters generally match as shown in FIG. 3B.

In an embodiment, the rod tapers outwardly between the second and third outer diameters. This tapering may add strength to the rod and prevent failure under high torque situations (as opposed to sudden changes in diameter such as 90-degree changes). In an embodiment, at least a portion of the rod tapers outwardly as it extends proximally. In an embodiment, at least a portion of the sleeve tapers outwardly as it extends proximally.

In an embodiment, a least a portion of the plurality of linear slots are included in a proximal half of the rod. By locating the slots near head 312, any fouling of the slots may be more quickly remedied by a clinician or manufacturer. For example, any particulate matter in the male/female keying mechanism (i.e., slots and projections) may be more quickly removed the more proximal the keying mechanism. Further, the slot/projection keying may serve to prevent rotation of the rod within the sleeve during implantation while also avoiding the manufacturing nuances needed for thread formation and/or the risk of cross-threading or fouling the threads with un-intended particulate matter and the like.

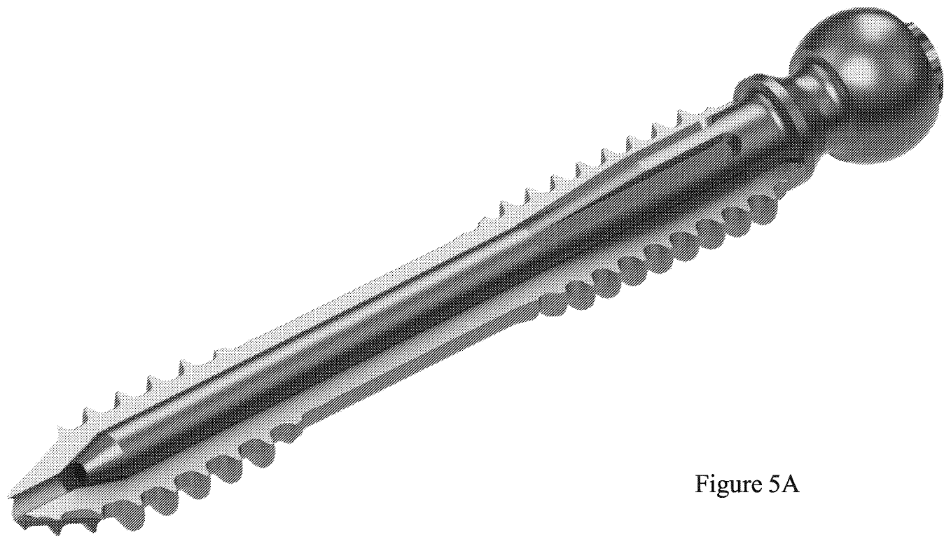

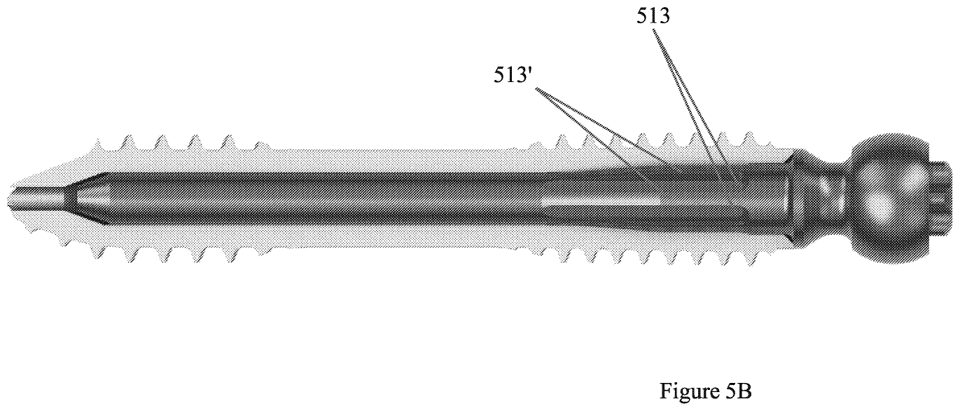

For example, FIGS. 5A, 5B include an embodiment with slots 513 and rod fins/male projections 513′ in the proximal half of the rod. As seen in FIG. 5B, each of the rod and the sleeve may be interpreted as having both male/female keyed portions that correspond with one another to resist rotation of the rod with respect to the sleeve.

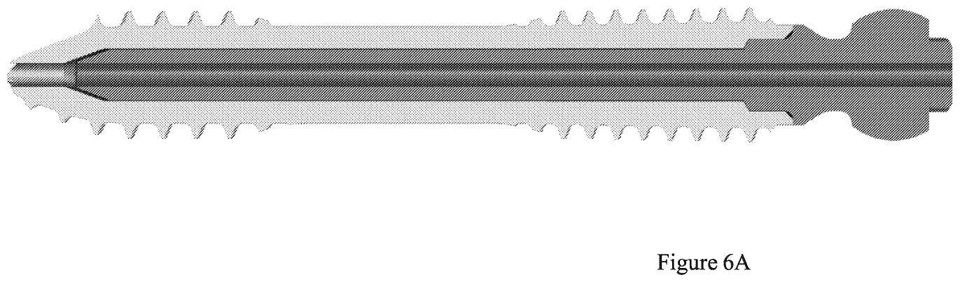

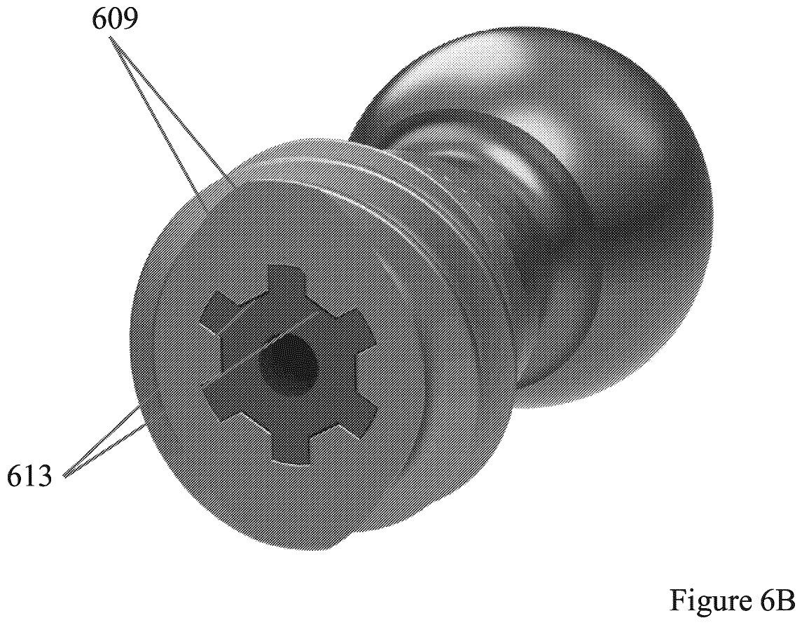

FIGS. 6A, 6B show an embodiment with more truncated keying elements located in the proximal half of the rod and sleeve. For example, see linear projections (609) on a wall of the central sleeve channel and linear slots 613 on an outer surface of the rod. This keyed arrangement is also present in the embodiment of FIG. 5A (although the structure of the sleeve inner wall is not as easily seen in that perspective).

An embodiment comprises a tulip configured to substantially surround the head of the rod. In an embodiment, the tulip includes a slot to slide around neck 317. However, in another embodiment the tulip may include resilient members which “pop” over head 312 and around neck 317. The tulip may be coupled to the head at a manufacturing facility and then shipped to a clinic or the tulip may be joined to the head by the clinician at the clinic.

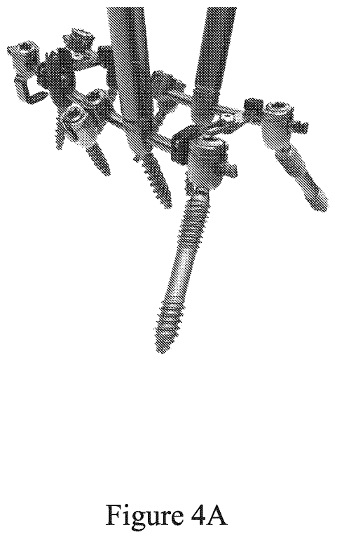

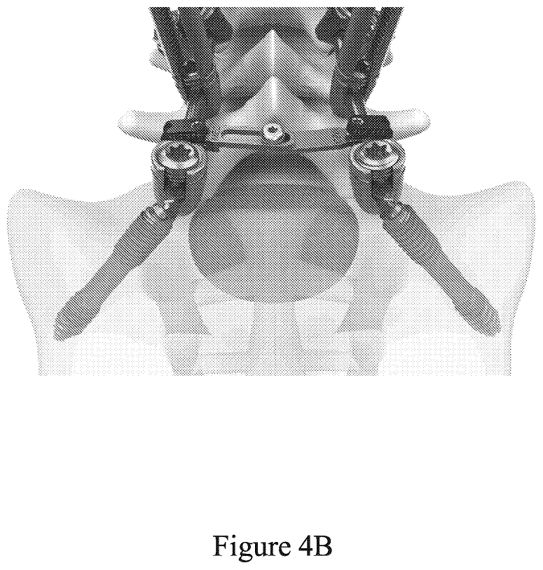

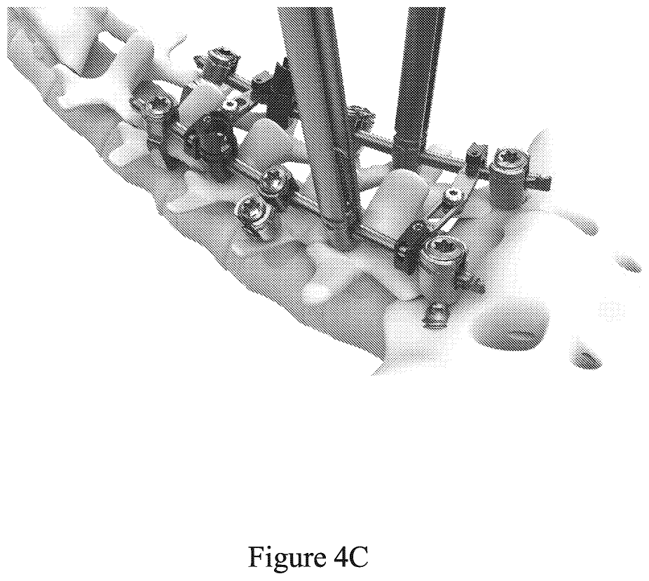

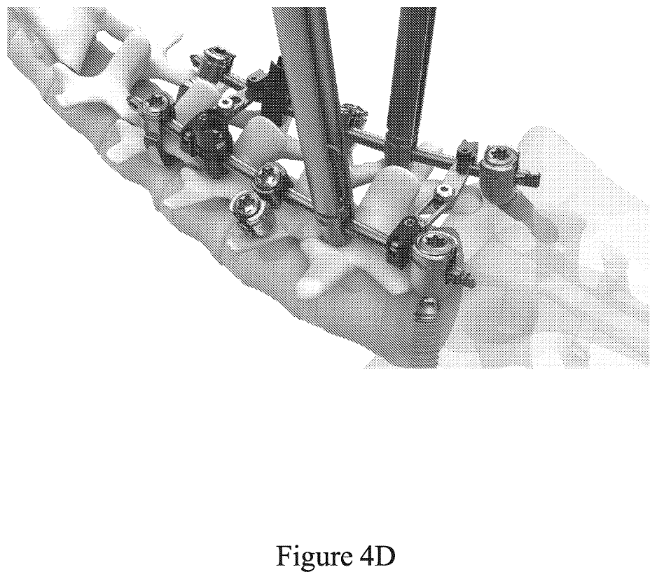

In an embodiment, the tulip may be used to couple the bone anchor system to a rod that affixes to additional tulips and bone anchors as shown in, for example, FIG. 4C. Such a system may promote better load distribution among the spine to increase the lifespan of the implants. In other words, the coupling of the SI anchor system to a vertebral anchor system may better distribute post-implant loading and lengthen the lifespan of the implant system and procedure.

In an embodiment the tulip includes a distal surface and a proximal surface. The proximal surface provides an opening to a trough, the through being configured to receive a fusion rod. The distal surface includes an aperture to encircle at least a portion of the head on the proximal end or the rod and/or a neck that couples the head of the rod to a main body of the rod. The proximal surface is coplanar with a first plane (320) and the distal surface is coplanar with a second plane (321). The first and second planes are not parallel with each other.

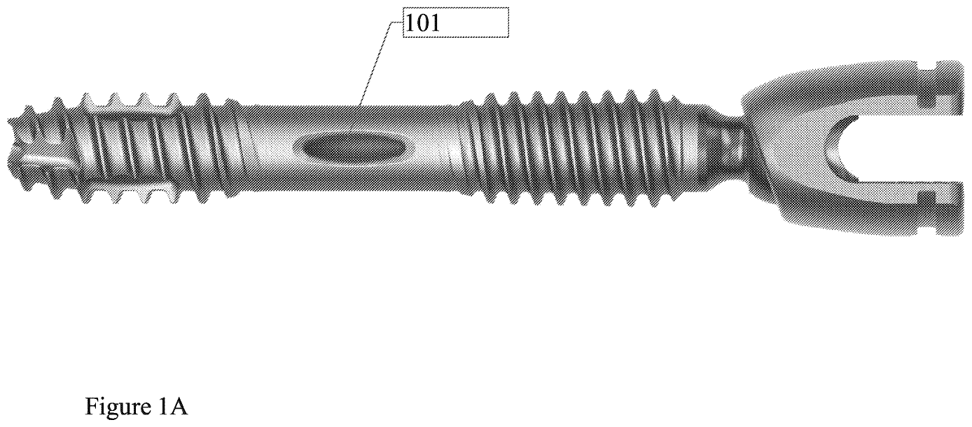

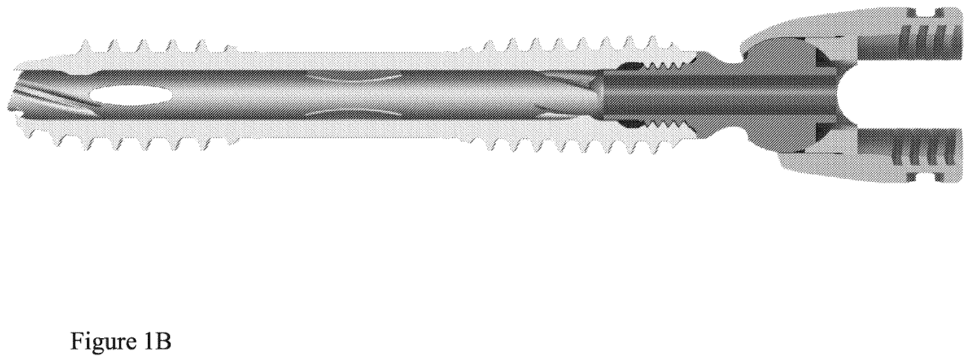

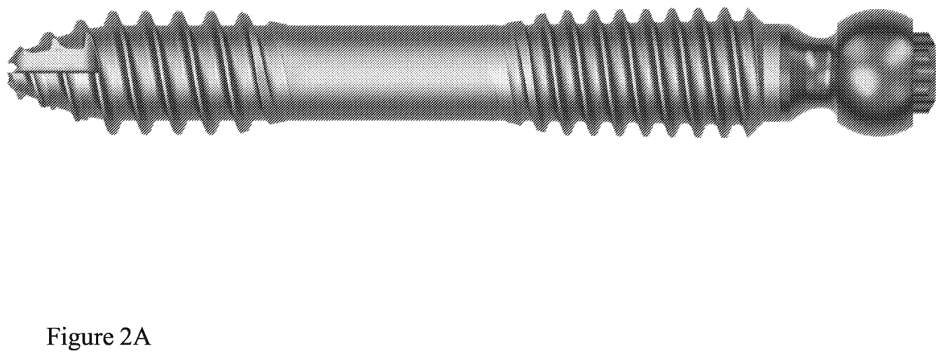

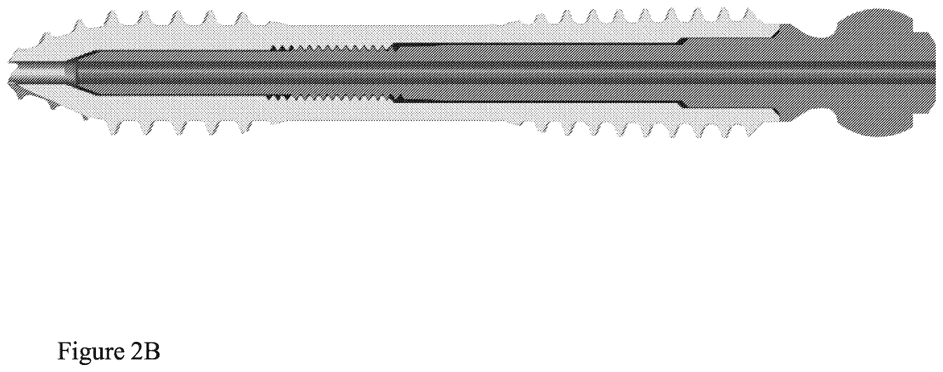

An embodiment includes a plurality of apertures that each couple the central sleeve channel to the outer surface of the sleeve. For an analogous aperture, see element 101 of FIG. 1A. For example, such an aperture may be included in region 307 to promote fusion along the SI joint. Region 307 is configured to be located at the SI joint in embodiments of some surgical techniques. In an embodiment, the plurality of apertures includes a first aperture and a second aperture. The first aperture includes a long axis parallel to the central sleeve axis. The first aperture includes a short axis orthogonal to the long axis of the first aperture. The first aperture includes a length measured along the long axis of the first aperture and a width measured along the short axis of the first aperture. The length of the first aperture is greater than the width of the first aperture.

In an embodiment, the sleeve is a sacroiliac bone anchor.

In an embodiment the first threaded portion includes a first thread pitch for a majority of threads of the first threaded portion. The second threaded portion includes a second thread pitch for a majority of threads of the second threaded portion. The first thread pitch is unequal to the second thread pitch. As used herein, thread pitch is measured from the peak of one thread to the peak of an immediately adjacent thread. In an embodiment, the first thread pitch is larger than the second thread pitch and the first threaded portion is distal to the second threaded portion.

In an embodiment, the outer sleeve is a compression screw. As used herein, a compression screw compresses two articles against one another when the screw is drilled across a seam that exists between the two articles. For example, differing thread pitches for areas 305, 306 result in a compression or lag screw that draws bone portions on either side of the SI joint towards each other in compression (i.e., one set of threads moves more quickly than another set of threads resulting in compression of adjacent bone portions). Smooth area 307 is configured to span the SI joint upon final implantation and its smoothness allows bone portions on either side of the SI joint to move/slide as fusion occurs over time.

In an embodiment, the bone anchor is a sacroiliac joint screw.

In an embodiment, the sleeve is a non-milled conduit and the rod is a milled conduit. However, in another embodiment the sleeve is a printed conduit.

In an embodiment, the sleeve includes projections on the outer surface of the sleeve and the projections are monolithic with the sleeve. As used herein, “monolithic” means formed from a single piece without use of adhesives, welds, or other coupling agents or means.

In an embodiment, the sleeve is welded to the rod. For example, at least one weld may be located at one or both of locations 318 (made via a hole formed in threads 306), 319 (at the most proximal sleeve/rod junction).

In an embodiment, the sleeve is permanently and non-slidingly coupled to the rod. For example, in an embodiment the sleeve is manufactured via a 3D printing process whereas the rod is manufactured via, for example, milling. As a result, the rod may have a higher strength with which to better tolerate torque and other forces experienced during implantation of the device and/or forces generated by the patient long after implantation. For example, high torque forces may be experienced at neck 317 during implantation of the system. However, the 3D process may allow for biocompatible surfaces that promote bone growth and better coupling between the implant and bone. For example, the surface of the sleeve may include irregular finger-like projections that are printed along with the rest of the sleeve with the projections and main conduit of the sleeve being monolithic with each other. Such projections may not be possible to form via a process such as milling.

In an embodiment, the sleeve is manufactured via additive manufacturing (e.g., 3D printing) while the rod is formed via milling. Then, at the manufacturing facility, the rod is forced into the sleeve and is retained within the sleeve via a resistance fit. In an embodiment, the rod is actually compressed radially by the sleeve. Further, the male/female locking portions 309, 313 resist rotation of the rod within the sleeve during, for example, insertion of the implant into the patient. The system may be shipped to a clinical facility with the rod already inserted into the sleeve. In fact, in an embodiment the sleeve and rod are permanently coupled to each other via at least one weld. The clinician may then receive the already coupled sleeve and rod from a sterile package and proceed to implant the sleeve and rod simultaneously. As a result, the benefits of additive manufacturing (e.g., biocompatible surface) are delivered along with the benefits of more robust manufacturing (e.g., milled rod) that has strength to tolerate high torque forced experienced during implantation.

In an embodiment, the central rod axis is coaxial with the central sleeve axis.

In an embodiment, the head of the rod includes a proximal face; the rod Includes a projection that projects proximally from the proximal face; the projection is configured to couple to an anchor driver. In an embodiment, the projection includes a side wall that extends proximally from the proximal face and parallel to the central rod axis; the proximal face is coplanar with third plane (324); and the side wall is orthogonal to the third plane. Such an arrangement of the proximal face and projection helps eliminate or lessen “screw toggle” where a screw or anchor is not rigidly affixed to a driver (e.g., screw driver) and therefore may include a long axis that is not collinear with a long axis of the driver. Screw toggle can be frustrating for clinicians trying to drive screws into bone a precise angle.

In an embodiment, the sleeve includes titanium and the rod includes titanium.

Further examples follow.

Example Set A: The following examples all pertain to the examples of Example Set A and not Example Set B.

Example 1. A bone anchor system comprising: a sleeve (301) that includes: (a) a central sleeve channel (302) that traverses the sleeve and extends from a proximal end (303) of the sleeve to a distal end (304) of the sleeve, (b) first (305) and second (306) threaded portions on an outer surface of the sleeve, (c) a non-threaded portion (307) on the outer surface of the sleeve and existing between the first and second threaded portions, (d) a central sleeve axis (308) that extends from the proximal end of the sleeve to the distal end of the sleeve, and (e) a plurality of linear projections (309) on a wall of the central sleeve channel; a rod (310) that includes: (a) a central rod channel (311) that traverses the rod and extends from a proximal end of the rod to a distal end of the rod, (b) a head (312) on the proximal end of the rod, the head including a circular cross-section; (c) a central rod axis that extends from the proximal end of the rod to the distal end of the rod, and (e) a plurality of linear slots (313) on an outer surface of the rod; wherein: (a) the rod is proportioned to slide within the central sleeve channel, and (b) the plurality of linear projections is proportioned to slide within the plurality of linear slots.

Alternative version of Example 1. A bone anchor system comprising: a sleeve (301) that includes: (a) a central sleeve channel (302) that traverses the sleeve and extends from a proximal end (303) of the sleeve to a distal end (304) of the sleeve, (b) first (305) and second (306) threaded portions on an outer surface of the sleeve, (c) a central sleeve axis (308) that extends from the proximal end of the sleeve to the distal end of the sleeve, and (e) a plurality of linear projections (309) on a wall of the central sleeve channel; a rod (310) that includes: (a) a central rod channel (311) that traverses the rod and extends from a proximal end of the rod to a distal end of the rod, (b) a head (312) on the proximal end of the rod, the head including a circular cross-section; (c) a central rod axis that extends from the proximal end of the rod to the distal end of the rod, and (e) a plurality of linear slots (313) on an outer surface of the rod; wherein: (a) the rod is proportioned to slide within the central sleeve channel, and (b) the plurality of linear projections is proportioned to slide within the plurality of linear slots.

Alternative version of Example 1. A bone anchor system comprising: a sleeve (301) that includes: (a) a central sleeve channel (302) that traverses the sleeve and extends from a proximal end (303) of the sleeve to a distal end (304) of the sleeve, (b) first (305) and second (306) threaded portions on an outer surface of the sleeve, (c) a non-threaded portion (307) on the outer surface of the sleeve and existing between the first and second threaded portions, (d) a central sleeve axis (308) that extends from the proximal end of the sleeve to the distal end of the sleeve, and (e) a plurality of linear projections (309) on a wall of the central sleeve channel; a rod (310) that includes: (a) a central rod channel (311) that traverses the rod and extends from a proximal end of the rod to a distal end of the rod, (b) a head (312) on the proximal end of the rod, the head including a circular cross-section; (c) a central rod axis that extends from the proximal end of the rod to the distal end of the rod, and (e) a plurality of linear slots (313) on an outer surface of the rod.

Example 2. The system of Example 1, wherein: a first of the plurality of the linear projections includes a first projection long axis that is parallel to the central sleeve axis; a second of the plurality of linear projections includes a second projection long axis that is parallel to the central sleeve axis.

Example 3. The system of Example 2, wherein: a first of the plurality of the linear slots includes a first slot long axis that is parallel to the central rod axis; a second of the plurality of linear slots includes a second slot long axis that is parallel to the central rod axis.

Example 4. The system according to any of Examples 1-3, wherein: the central sleeve channel includes first (314), second (315), and third (316) inner diameters; the first inner diameter is distal to the second and third inner diameters and the second inner diameter is between the first and third inner diameters; the second inner diameter is less than the third inner diameter.

Example 5. The system of Example 4, wherein the first inner diameter is less than the second inner diameter.

Example 6. The system according to any of Examples 1-5, wherein: the rod includes first, second, and third outer diameters; the first outer diameter is distal to the second and third outer diameters and the second outer diameter is between the first and third outer diameters; the second outer diameter is less than the third outer diameter.

Example 7. The system of Example 6, wherein the first outer diameter is less than the second outer diameter.

Example 7.1 The system according to any of Examples 6 to 7, wherein the rod tapers outwardly between the second and third outer diameters.

Example 8. The system according to any of Examples 1 to 7, wherein the a least a portion of the plurality of linear slots are included in a proximal half of the rod.

Example 9. The system according to any of Examples 1 to 8 comprising a tulip configured to substantially surround the head of the rod.

Example 9.1 The system of Example 9, wherein: the tulip Includes a distal surface and a proximal surface; the proximal surface provides an opening to a trough, the through being configured to receive a fusion rod; the distal surface includes an aperture to encircle at least a portion of the head on the proximal end, a neck that couple the head of the rod to a main body of the rod; the proximal surface is coplanar with a first plane (320) and the distal surface is coplanar with a second plane (321); the first and second planes are not parallel with each other.

Example 10. The system according to any of Examples 1 to 9 comprising a plurality of apertures that each couple the central sleeve channel to the outer surface of the sleeve,

Example 11. The system according to any of Examples 1-10, wherein: the plurality of apertures includes a first aperture and a second aperture; the first aperture includes a long axis parallel to the central sleeve axis; the first aperture includes a short axis orthogonal to the long axis of the first aperture; the first aperture includes a length measured along the long axis of the first aperture and a width measured along the short axis of the first aperture; the length of the first aperture is greater than the width of the first aperture.

Example 12. The system according to any of Examples 1 to 11, wherein the sleeve is a sacroiliac bone anchor.

Example 13. The system according to any of Examples 1 to 12, wherein; the first threaded portion includes a first thread pitch for a majority of threads of the first threaded portion; the second threaded portion includes a second thread pitch for a majority of threads of the second threaded portion; the first thread pitch is unequal to the second thread pitch.

Example 14. The system of Example 13, wherein: the first thread pitch is larger than the second thread pitch; the first threaded portion is distal to the second threaded portion.

Example 15. The system of Example 14, wherein the outer sleeve is a compression screw.

Example 15.1 The system according to any of Examples 1 to 15, wherein the bone anchor is a sacroiliac joint screw.

Example 16. The system according to any of Examples 1 to 15, wherein; the sleeve is a non-milled conduit; the rod is a milled conduit.

Example 17. The system according to any of Examples 1 to 16, wherein the sleeve is a printed conduit.

Example 18. The system according to any of Examples 1 to 17, wherein: the sleeve Includes projections on the outer surface of the sleeve; the projections are monolithic with the sleeve.

Example 19. The system according to any of Examples 1 to 18, wherein the sleeve is welded to the rod.

Example 20. The system according to any of Examples 1 to 18, wherein the sleeve is permanently and non-slidingly coupled to the rod.

Example 21. The system according to any of Examples 1 to 20, wherein the central rod axis is coaxial with the central sleeve axis.

Example 22. The system according to any of Examples 1 to 21, wherein; the head of the rod includes a proximal face; the rod includes a projection that projects proximally from the proximal face; the projection is configured to couple to an anchor driver.

Example 23. The system of Example 22, wherein: the projection includes a side wall that extends proximally from the proximal face and parallel to the central rod axis; the proximal face is coplanar with third plane (324); the side wall is orthogonal to the third plane.

Example 24. The system according to any of Examples 1 to 23, wherein: the sleeve includes titanium; the rod includes titanium.

Example 25. A bone anchor system comprising: a first conduit that includes: (a) a central first conduit channel that traverses the first conduit and extends from a proximal end of the first conduit to a distal end of the first conduit, (b) first and second threaded portions on an outer surface of the first conduit, (c) a non-threaded portion on the outer surface of the first conduit and existing between the first and second threaded portions, (d) a central first conduit axis that extends from the proximal end of the first conduit to the distal end of the first conduit, and (e) a first keyed element on a wall of the central first conduit channel; a second conduit that includes: (a) a central second conduit channel that traverses the second conduit and extends from a proximal end of the second conduit to a distal end of the second conduit, (b) a head on the proximal end of the second conduit, the head including a circular cross-section; (c) a central second conduit axis that extends from the proximal end of the second conduit to the distal end of the second conduit, and (e) a second keyed element on an outer surface of the rod; wherein second key element is complementary with the first keyed element.

Example Set B: The following examples all pertain to the examples of Example Set B and not Example Set A.

Example 1. A bone anchor system comprising: a sleeve (701) that includes: (a) a central sleeve channel (702) that traverses the sleeve and extends from a proximal end (703) of the sleeve to a distal end (704) of the sleeve, (b) a threaded first portion (705) and a threaded second portion (706) on an outer surface of the sleeve, (c) a third portion (707) on the outer surface of the sleeve and existing between the first and second portions, (d) a central sleeve axis (708) that extends from the proximal end of the sleeve to the distal end of the sleeve, and (e) a plurality of threads (709) on a wall of the central sleeve channel; a rod (710) that includes: (a) a central rod channel (711) that traverses the rod and extends from a proximal end of the rod to a distal end of the rod, (b) a head (712) on the proximal end of the rod, the head including a circular cross-section; (c) a central rod axis that extends from the proximal end of the rod to the distal end of the rod, and (e) a plurality of threads (713) on an outer surface of the rod; wherein: (a) the rod is proportioned to slide within the central sleeve channel, and (b) the plurality of threads on the outer surface of the rod are configured to screw into the plurality of threads on the wall of the central sleeve channel.

Alternative version of Example 1. A bone anchor system comprising: a sleeve (701) that includes: (a) a central sleeve channel (702) that traverses the sleeve and extends from a proximal end (703) of the sleeve to a distal end (704) of the sleeve, (b) threaded first (705) and second (706) portions on an outer surface of the sleeve, (c) a central sleeve axis (708) that extends from the proximal end of the sleeve to the distal end of the sleeve, and (e) a plurality of threads (709) on a wall of the central sleeve channel; a rod (710) that includes: (a) a central rod channel (711) that traverses the rod and extends from a proximal end of the rod to a distal end of the rod, (b) a head (712) on the proximal end of the rod, the head including a circular cross-section; (c) a central rod axis that extends from the proximal end of the rod to the distal end of the rod, and (e) a plurality of threads (713) on an outer surface of the rod; wherein: (a) the rod is proportioned to slide within the central sleeve channel, and (b) the plurality of threads on the outer surface of the rod are configured to screw into the plurality of threads on the wall of the central sleeve channel.

For example, some embodiments may extend threads 705 or 706 into areas 707. Other embodiments may include a set of threads at area 707 with a pitch that differs from the pitch of threads of either or both of threads 705, 706. Fenestrations (i.e., apertures, holes) (751, 752) may be included in smooth or threaded areas of the sleeve and/or rod. In some embodiments, fenestrations are included in the sleeve but not the rod such that an axis (753,753′), which is orthogonal to a long axis of the sleeve, traverses the aperture but not the rod (because the rod does not extend distally to the point of the aperture) to provide a path for bone fusion/growth.

Alternative version of Example 1. A bone anchor system comprising: a sleeve (701) that includes: (a) a central sleeve channel (702) that traverses the sleeve and extends from a proximal end (703) of the sleeve to a distal end (704) of the sleeve, (b) threaded first (705) and second (706) portions on an outer surface of the sleeve, (c) a central sleeve axis (708) that extends from the proximal end of the sleeve to the distal end of the sleeve, and (e) a plurality of threads (709) on a wall of the central sleeve channel; a rod (710) that includes: (a) a central rod channel (711) that traverses the rod and extends from a proximal end of the rod to a distal end of the rod, (b) a head (712) on the proximal end of the rod, the head including a circular cross-section; (c) a central rod axis that extends from the proximal end of the rod to the distal end of the rod, and (e) a plurality of threads (713) on an outer surface of the rod.

Example 2. The system of Example 1, wherein: the sleeve includes first (751) and second (752) apertures in a sidewall of the sleeve; an axis (753), which is orthogonal to the long axis of the sleeve, traverses the first and second apertures but not the rod.

For example, such an aperture may be included in region 707 to promote fusion along the SI joint. Region 707 is configured to be located at the SI joint in embodiments of some surgical techniques. In an embodiment, the first and second apertures are in a middle third of the sleeve. This positioning helps position the apertures across, for example, a fracture between two bone pieces and allow for fusion between the two bone pieces.

Example 3. The system of Example 1, wherein: the sleeve includes first and second apertures in a sidewall of the sleeve; the first aperture includes a long axis (754) parallel to the central sleeve axis; the first aperture includes a short axis orthogonal to the long axis of the first aperture; the first aperture includes a length measured along the long axis of the first aperture and a width measured along the short axis of the first aperture; the length of the first aperture is greater than the width of the first aperture.

Example 4. The system according to any of Examples 1-3, wherein: the central sleeve channel includes first (714), second (715), and third (716) inner diameters; the first inner diameter is distal to the second and third inner diameters and the second inner diameter is between the first and third inner diameters; the second inner diameter is less than the third inner diameter.

Example 5. The system of Example 4, wherein the first inner diameter is less than the second inner diameter.

Example 6. The system according to any of Examples 4-5, wherein a portion (760) of the central sleeve channel tapers outwardly as it extends proximally.

Example 7. The system of Example 6, wherein: a portion of the rod tapers outwardly as it extends proximally; a transverse axis is orthogonal to the central sleeve axis and intersects both the portion (760) of the central sleeve and the portion of the rod

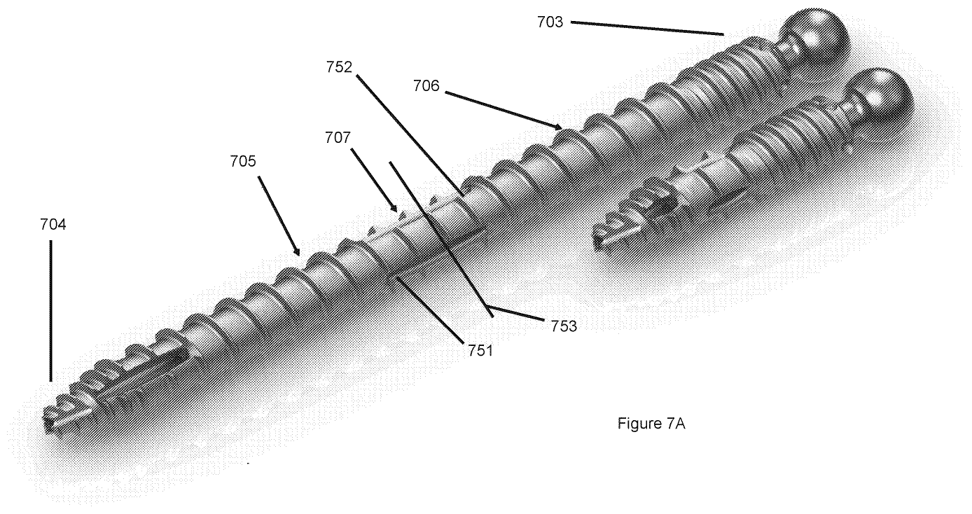

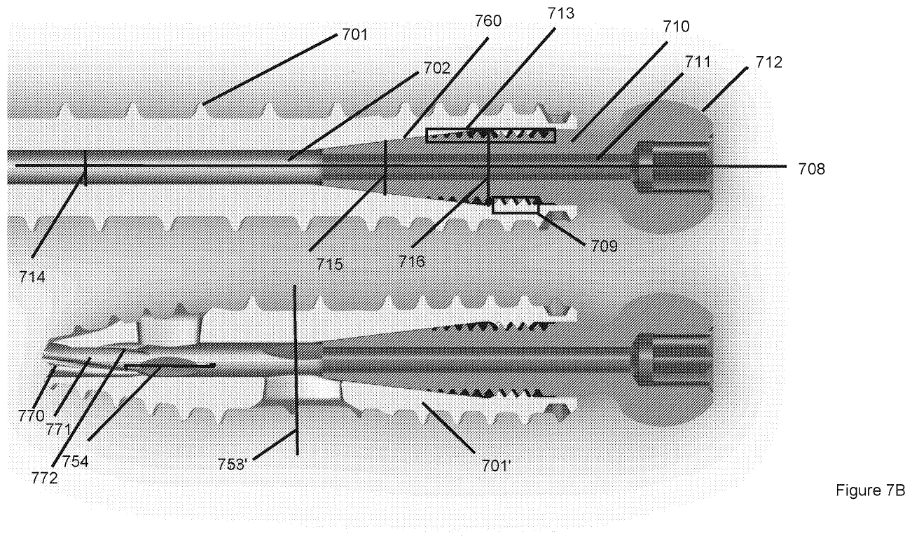

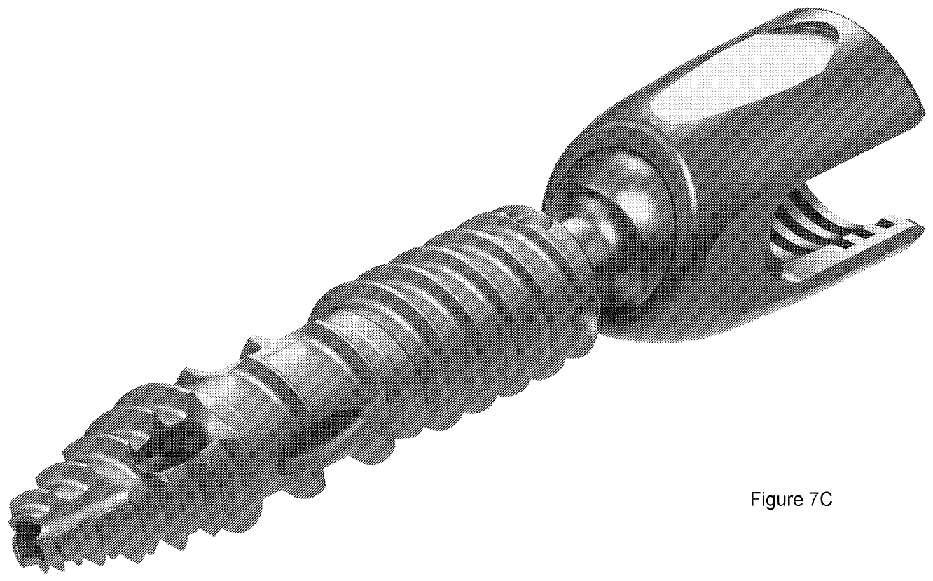

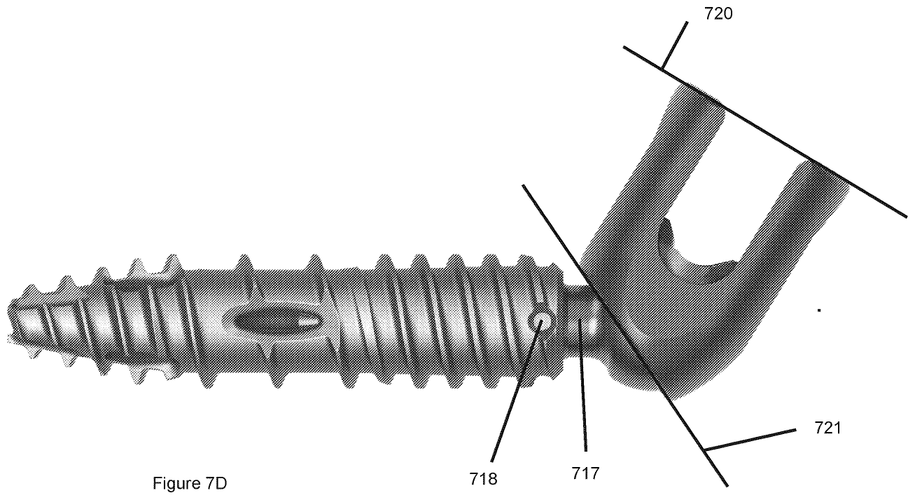

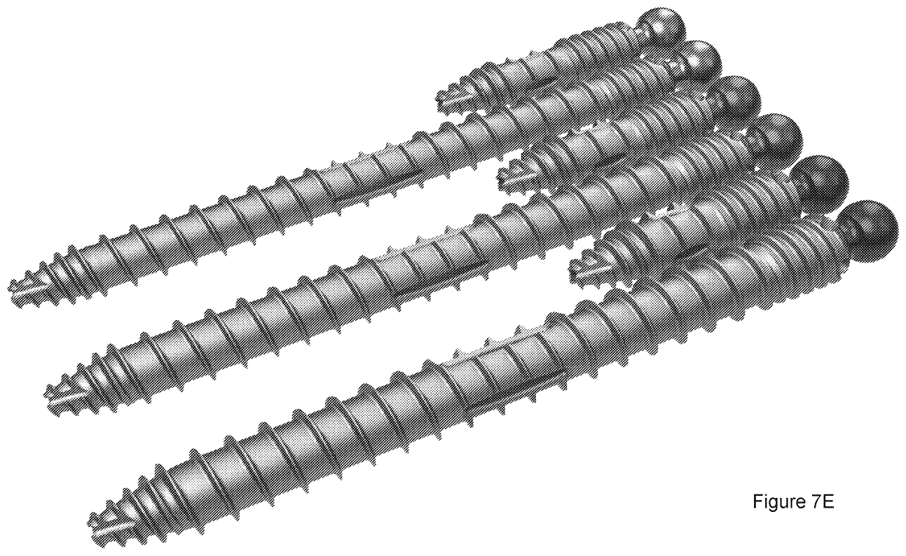

For example, the transverse axis may align with diameter 715 of FIG. 7B. This tapering may add strength to the rod and prevent failure under high torque situations (as opposed to sudden changes in diameter such as 90-degree changes). In an embodiment, at least a portion of the rod tapers outwardly as it extends proximally. In an embodiment, at least a portion of the sleeve tapers outwardly as it extends proximally.

Example 8. The system according to any of Examples 1-3, wherein: the rod includes first, second, and third outer diameters; the first outer diameter is distal to the second and third outer diameters and the second outer diameter is between the first and third outer diameters; the second outer diameter is less than the third outer diameter.

In an embodiment, the first outer diameter is less than the second outer diameter. However, in other embodiments the diameter relationships are not so limited and may include more or fewer distinct outer diameters for the rod. For example, diameter 1 (714) may remain but diameter 2 (715) may extend proximally to end 703. In an embodiment, at least some of the inner diameters for the sleeve correspond to outer diameters for the rod. In other words, at least some of the inner and outer diameters generally match as shown in FIG. 7B.

Example 8.1 The system according to any of Examples 1 to 7, wherein the plurality of threads on the wall of the central sleeve channel are included entirely in a proximal third of the sleeve.

Example 9. The system according to any of Examples 1 to 8 comprising a tulip configured to substantially surround the head of the rod.

In an embodiment, the tulip includes a slot to slide around neck 717. However, in another embodiment the tulip may include resilient members which “pop” over head 712 and around neck 717. In another embodiment, the rod is slid through the distal aperture of the tulip and then into the sleeve. Afterwards, the rod and sleeve may be welded together at location 718 or may otherwise be fixedly coupled to each other and an interface between the rod and sleeve. The tulip may be coupled to the head at a manufacturing facility and then shipped to a clinic or the tulip may be joined to the head by the clinician at the clinic.

In an embodiment, the tulip may be used to couple the bone anchor system to a rod that affixes to additional tulips and bone anchors. Such as system may promote better load distribution among the spine to increase the lifespan of the implants. In other words, the coupling of the SI anchor system to vertebral anchor system may better distribute post-implant loading and lengthen the lifespan of the implant system and procedure.

Example 10. The system of Example 9, wherein: the tulip includes a distal surface and a proximal surface; the proximal surface provides an opening to a trough, the through being configured to receive a fusion rod; the distal surface includes an aperture to encircle at least a portion of the rod; the proximal surface is coplanar with a first plane (720) and the distal surface is coplanar with a second plane (721); the first and second planes are not parallel with each other.

Example 11. The system according to any of Examples 1-10, wherein the third portion is threaded.

Example 12. The system according to any of Examples 1 to 11, wherein the sleeve is a sacroiliac bone anchor.

Example 13. The system according to any of Examples 1 to 12, wherein: the first portion includes a first thread pitch for a majority of threads of the first portion; the second portion includes a second thread pitch for a majority of threads of the second portion; the first thread pitch is unequal to the second thread pitch.

As used herein, thread pitch is measured from the peak of one thread to the peak of an immediately adjacent thread,

Example 14. The system of Example 13, wherein: the first thread pitch is larger than the second thread pitch; the first threaded portion is distal to the second threaded portion.

Example 15. The system of Example 14, wherein the outer sleeve is a compression screw.

As used herein, a compression screw compresses two articles against one another when the screw is drilled across a seam that exists between the two articles.

For example, differing thread pitches for areas 705, 706 result in a compression or lag screw that draws bone portions on either side of the SI joint towards each other in compression (i.e., one set of threads moves more quickly than another set of threads resulting in compression of adjacent bone portions). Smooth area 707 is configured to span the SI joint upon final implantation and its smoothness allows bone portions on either side of the SI joint to move/slide as fusion occurs over time.

Example 16. The system according to any of Examples 1 to 15, wherein: the sleeve is a non-milled conduit; the rod is a milled conduit.

Example 17. The system according to any of Examples 1 to 16, wherein the sleeve is a printed conduit.

Example 18. The system according to any of Examples 1 to 17, wherein: the sleeve includes projections on the outer surface of the sleeve; the projections are monolithic with the sleeve.

As used herein, “monolithic” means formed from a single piece without use of adhesives, welds, or other coupling agents or means.

Example 19. The system according to any of Examples 1 to 18, wherein the sleeve is welded to the rod.

For example, at least one weld may be located at location 718 (made via a hole formed in threads 706).

Example 20. The system according to any of Examples 1 to 18, wherein the sleeve is permanently and non-slidingly coupled to the rod.

For example, in an embodiment the sleeve is manufactured via a 3D printing process whereas the rod is manufactured via, for example, milling. As a result, the rod may have a higher strength with which to better tolerate torque and other forces experienced during implantation of the device and/or forces generated by the patient long after implantation. For example, high torque forces may be experienced at neck 717 during implantation of the system. However, the 3D process may allow for biocompatible surfaces that promote bone growth and better coupling between the implant and bone. For example, the surface of the sleeve may include irregular finger-like projections that are printed along with the rest of the sleeve with the projections and main conduit of the sleeve being monolithic with each other. Such projections may not be possible to form via a process such as milling.

In an embodiment, the sleeve is manufactured via additive manufacturing (e.g., 3D printing) while the rod is formed via milling. Then, at the manufacturing facility, the rod is forced into the sleeve and is retained within the sleeve via a resistance fit. In an embodiment, the rod is actually compressed radially by the sleeve. The system may be shipped to a clinical facility with the rod already inserted into the sleeve. In fact, in an embodiment the sleeve and rod are permanently coupled to each other via at least one weld. The clinician may then receive the already coupled sleeve and rod from a sterile package and proceed to implant the sleeve and rod simultaneously. As a result, the benefits of additive manufacturing (e.g., biocompatible surface) are delivered along with the benefits of more robust manufacturing (e.g., milled rod) that has strength to tolerate high torque forced experienced during implantation.

Example 21. The system according to any of Examples 1 to 20, wherein the central rod axis is coaxial with the central sleeve axis.

Example 22. The system according to any of Examples 1 to 21 comprising: an additional sleeve (701′) that includes: (a) an additional central sleeve channel that traverses the additional sleeve and extends from a proximal end of the additional sleeve to a distal end of the additional sleeve, (b) an additional threaded first portion and an additional threaded second portion on an outer surface of the additional sleeve, (c) an additional third portion on the outer surface of the additional sleeve and existing between the additional first and second portions, (d) an additional central sleeve axis that extends from the proximal end of the additional sleeve to the distal end of the additional sleeve, and (e) an additional plurality of threads on a wall of the additional central sleeve channel; wherein: (a) the rod is proportioned to slide within the additional central sleeve channel, and (b) the plurality of threads on the outer surface of the rod are configured to screw into the additional plurality of threads on the wall of the additional central sleeve channel.

As shown in FIG. 7B, a single rod (or identical rods) can be used for sleeves of varying lengths. This decreases the amount of inventory needed to properly prepare of surgeries that may require short, long, or short and long outer sleeves.

Example 23. The system according to any of Examples 1 to 22, wherein: the sleeve includes titanium; the rod includes titanium.

Example 24. The system according to any of Examples 1-23, wherein an internal wall of the central sleeve channel includes a first ridge (770), a second ridge (772), and a trough (771) between the first and second ridges.

Such a trough may help funnel or direct bone particulate (e.g., created via drilling the sleeve or a wire) away from the distal entrance to the sleeve channel and towards apertures (e.g., aperture 751) to help promote bone fusion.

While various embodiments described herein address fixating a SI joint, other embodiments are not so limited and may be used generally to couple tissues together, such as two portions of bone or other tissue. Those portions need not be on opposite sides of a joint. For example, an anchor embodiment described herein could be used as a shank of a pedicle screw. Other embodiments may be used for surgery of the shoulder, wrist, toe (i.e., anywhere an implant is placed where bone is directed from one location to the internal flute of the implant to facilitate fusion).

The foregoing description of the embodiments of the invention has been presented for the purposes of Illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a certain side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first article “on” a second article is directly on and in immediate contact with the second article unless such is specifically stated; there may be a third article or other structure between the first article and the second article on the first article. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

1. A bone anchor system comprising:
a sleeve that includes: (a) a central sleeve channel that traverses the sleeve and extends from a proximal end of the sleeve to a distal end of the sleeve, (b) a threaded first portion and a threaded second portion on an outer surface of the sleeve, (c) a third portion on the outer surface of the sleeve and existing between the first and second portions, (d) a central sleeve axis that extends from the proximal end of the sleeve to the distal end of the sleeve, and (e) a plurality of threads on a wall of the central sleeve channel;
a rod that includes: (a) a central rod channel that traverses the rod and extends from a proximal end of the rod to a distal end of the rod, (b) a head on the proximal end of the rod, the head including a circular cross-section; (c) a central rod axis that extends from the proximal end of the rod to the distal end of the rod, and (e) a plurality of threads on an outer surface of the rod;
wherein: (a) the rod is proportioned to slide within the central sleeve channel, and (b) the plurality of threads on the outer surface of the rod are configured to screw into the plurality of threads on the wall of the central sleeve channel.

2. The system of claim 1, wherein:
the sleeve includes first and second apertures in a sidewall of the sleeve;
an axis which is orthogonal to the central sleeve axis, traverses the first and second apertures but not the rod.

3. The system of claim 1, wherein:
the sleeve includes first and second apertures in a sidewall of the sleeve;
the first aperture includes a long axis parallel to the central sleeve axis;
the first aperture includes a short axis orthogonal to the long axis of the first aperture;
the first aperture includes a length measured along the long axis of the first aperture and a width measured along the short axis of the first aperture;
the length of the first aperture is greater than the width of the first aperture.

4. The system according to claim 3, wherein:
the central sleeve channel includes first, second, and third inner diameters;
the first inner diameter is distal to the second and third inner diameters and the second inner diameter is between the first and third inner diameters;
the second inner diameter is less than the third inner diameter.

5. The system of claim 4, wherein the first inner diameter is less than the second inner diameter.

6. The system according to claim 5, wherein a portion of the central sleeve channel tapers outwardly as it extends proximally.

7. The system of claim 6, wherein:
a portion of the rod tapers outwardly as it extends proximally;
a transverse axis is orthogonal to the central sleeve axis and intersects both the portion of the central sleeve channel and the portion of the rod.

8. The system according to claim 7, wherein the plurality of threads on the wall of the central sleeve channel are included entirely in a proximal third of the sleeve.

9. The system according to claim 7 comprising a tulip configured to substantially surround the head of the rod.

10. The system of claim 9, wherein:
the tulip includes a distal surface and a proximal surface;
the proximal surface provides an opening to a trough, the trough being configured to receive a fusion rod;
the distal surface includes an aperture to encircle at least a portion of the rod;
the proximal surface is coplanar with a first plane and the distal surface is coplanar with a second plane;
the first and second planes are not parallel with each other.

11. The system according to claim 10, wherein the third portion is threaded.

12. The system according to claim 11, wherein the sleeve is a sacroiliac bone anchor.

13. The system according to claim 12, wherein:
the first portion includes a first thread pitch for a majority of the threads of the first portion;
the second portion includes a second thread pitch for a majority of the threads of the second portion;
the first thread pitch is unequal to the second thread pitch.

14. The system of claim 13, wherein:
the first thread pitch is larger than the second thread pitch;
the first threaded portion is distal to the second threaded portion.

15. The system of claim 14, wherein the sleeve is a compression screw.

16. The system according to claim 15, wherein:
the sleeve is a non-milled conduit;
the rod is a milled conduit.

17. The system according to claim 16, wherein the sleeve is a printed conduit.

18. The system according to claim 17, wherein:
the sleeve includes projections on the outer surface of the sleeve;
the projections are monolithic with the sleeve.

19. The system according to claim 18, wherein the sleeve is welded to the rod.

20. The system according to claim 18, wherein the sleeve is permanently and non-slidingly coupled to the rod.

21. The system according to claim 20, wherein the central rod axis is coaxial with the central sleeve axis.

22. The system according to claim 21 comprising:
an additional sleeve that includes: (a) an additional central sleeve channel that traverses the additional sleeve and extends from a proximal end of the additional sleeve to a distal end of the additional sleeve, (b) an additional threaded first portion and an additional threaded second portion on an outer surface of the additional sleeve, (c) an additional third portion on the outer surface of the additional sleeve and existing between the additional first and second portions, (d) an additional central sleeve axis that extends from the proximal end of the additional sleeve to the distal end of the additional sleeve, and (e) an additional plurality of threads on a wall of the additional central sleeve channel;
wherein: (a) the rod is proportioned to slide within the additional central sleeve channel, and (b) the plurality of threads on the outer surface of the rod are configured to screw into the additional plurality of threads on the wall of the additional central sleeve channel.

23. The system according to claim 22, wherein:
the sleeve includes titanium;
the rod includes titanium.

24. The system according to claim 23, wherein an internal wall of the central sleeve channel includes a first ridge, a second ridge-(772), and a trough between the first and second ridges.
